(19) United States
(12) Patent Application Publication
(10) Pub. No.: US 2021/0161770 A1
(43) Pub. Date: Jun. 3, 2021

(54) WATER-HARDENING DENTAL CEMENT, METHOD AND KIT FOR PRODUCING THE SAME AND USE THEREOF

(71) Applicant: Muhlbauer Technology GmbH, Hamburg (DE)

(72) Inventors: Henrik Bottcher, Tostedt (DE); Stephan Neffgen, Pinneberg (DE); Holger Muller, Pinneberg (DE)

(21) Appl. No.: 17/041,311

(22) PCT Filed: Jul. 12, 2018
(86) PCT No.: PCT/EP2018/068948

§ 371 Date: Feb. 9, 2021

(30) Foreign Application Priority Data
Mar. 27, 2018 (DE) 10 2018 204 655.7

(51) Int. Cl. A61K 6/86; A61C 5/00; A61K 6/893; A61K 6/891; A61K 6/17; A61K 6/838

(52) U.S. Cl. CPC A61K 6/86; A61C 5/00; A61K 6/893; A61C 13/082; A61K 6/17; A61K 6/838; A61K 6/891

(57) ABSTRACT

The invention provides a water-hardening dental cement comprising an acid-reactive powder, a polyprotic acid, water and dispersed polymer particles.

The invention further provides a method for producing a water-hardening dental cement, a kit for producing a water-hardening dental cement and a use of the water-hardening dental cement.

The invention relates to a water-hardening dental cement, to a method for producing a water-hardening dental cement, to a kit for producing a water-hardening dental cement and to the use thereof as a dental filling and/or luting material.

Considered from a chemical viewpoint, water-hardening dental cements are substances which on mixing powder and liquid set via an acid-base reaction. For this purpose, the dental cements comprise at least three constituents: an acid-reactive powder as a base, an acid and water.

Conventional glass ionomer cements (CGIC) are water-hardening cements which essentially comprise the following three constituents: acid-reactive glass powder, a polyalkenoic acid and water. The powder contains, by way of example, an acid-reactive fluoroaluminosilicate glass. The liquid contains water. The acid is usually a water-soluble polyalkenoic acid, which may be present either in the powder or in the liquid.

An example of a conventional glass ionomer cement for fillings (cavity classes III and V, root caries, tunnel preparations, milk teeth cavities and underfillings) known in the prior art is sold under the name Alpha® Fil (DMG). Further known products are GC Fuji IX® and Ketac® Fil.

The critical stress intensity factor K_{Ic} is a measure of the fracture toughness of a material. The problem with known cements is that they have only an inadequate fracture toughness. The present invention is intended to solve this problem.

The prior art aims to improve the fracture toughness of water-containing dental cements by the addition of polymerizable components, in particular by the addition of (meth)acrylate group-comprising monomers, polymers and/or fillers. However, such polymerizable cements are associated with disadvantages compared to the conventional glass ionomer cements, for example a reduced biocompatibility.

JP 2001354509 describes a powder mixture for glass ionomer cements having improved mechanical properties, especially fracture toughness, and containing fibers based on apatite or fluoroapatite. The fibers bring about anisotropic reinforcement. However, a disadvantage of the fibers used is that they can generate an undesirably rough surface.

U.S. Pat. No. 6,860,932 describes an improvement in the fracture toughness by means of TiO₂ or Al₂O₃ particles. The metal oxides described have a high index of refraction and therefore increase the opacity of the glass ionomer cement, which makes a tooth-like coloring more difficult.

WO 2017083039 A1 discloses a kit for producing a glass ionomer cement which comprises alumina- or silica-based particles as one constituent.

It is an object of the invention to provide a water-hardening dental cement which has an improved fracture toughness without at the same time impairing otherwise advantageous properties.

This object is achieved by a water-hardening dental cement according to the main claim. The invention thus relates in particular to a water-hardening dental cement comprising an acid-reactive powder, a polyprotic acid, water and dispersed polymer particles.

Further advantageous embodiments can be found in the subclaims.

First of all, some terms employed within the context of the invention will be explained.

The critical stress intensity factor K_{Ic} is a measure of the fracture toughness of a material. The stress intensity factor K is a measure of the intensity of the stress field in the vicinity of a crack tip. The stress intensity factor at which a fracture ultimately occurs is the critical stress intensity factor. This material property is also referred to as the fracture toughness. The critical stress intensity factor K_{IC} characterizes the fracture toughness when a crack opens perpendicularly to the crack surface. This crack opening mode has the greatest significance in practice.

An “acid-reactive powder” is understood within the context of the invention to mean a powder that reacts with a polyprotic acid via an acid-base reaction. Suitable acid-reactive powders are known in the prior art and are explained in more detail hereinbelow.

A “polyprotic acid” is understood in the prior art and within the context of the invention to mean an acid which has a plurality of protons and in which the release of protons occurs in steps over a plurality of dissociation stages. Polyprotic acids suitable for the invention are explained in more detail hereinbelow.

The essence of the invention is to provide a water-hardening dental cement, preferably a conventional glass ionomer cement, with polymer particles dispersed therein, as a result of which a crack propagation in the cement which results in fracture is impeded. The fracture toughness of the water-hardening cement is thus improved in a simple manner and without degradation of other properties. The increase in the fracture toughness or the critical stress intensity factor K_{Ic} of the water-hardening cement in turn results in an increase in the durability of a filling and can enable a extension of indication, in particular to masticatory load-bearing fillings.

Therefore, within the context of the invention, the fracture toughness or the critical stress intensity factor K_{Ic} is improved while retaining or improving the other advantageous properties of the water-hardening dental cements. Examples of other properties of water-hardening dental cement, in particular conventional glass ionomer cement, that should be taken into account are consistency/moldability, working time, setting time (1.5-6 min), compressive strength (>100, >180 MPa), flexural strength (>25 MPa), abrasion/acid erosion (<0.17 mm), optical properties (opacity CGIC C0.70 of 0.35-0.90), X-ray visibility (=% Al), shade and color stability, self-adhesion/moisture tolerance, biocompatibility/sensitivities, fluoride release and expansion behavior. The minimum requirements specified for conventional glass ionomer cement are taken from ISO9917.

Further advantages of the invention are that the polymer particles of the invention are toxicologically harmless and only make up a very low proportion by weight in the cement. The main components in the dental cement selected can further be commercially available and clinically proven cement constituents. In particular, the addition of polymerizable compounds such as (meth)acrylates can also be dispensed with.

Within the context of the invention, it is preferable for the water-hardening dental cement to be a glass ionomer cement, more preferably a conventional glass ionomer cement (CGIC).

Conventional glass ionomer cements are known in the prior art; they cure via an acid-base reaction. In addition to the conventional glass ionomer cements, there are also, for example, what are called metal-reinforced glass ionomer cements or cermet cements (e.g. Ketac® Silver and Alpha® Silver) and plastic-modified glass ionomer cements (e.g. Photac® Fil Quick, Vitremer® Fuji® II LC). One advantage of the conventional glass ionomer cements is their good chemical adhesion to hard tooth tissues, for example.

### Polymer Particles

Suitable polymer particles form a polymer dispersion in water or in an aqueous phase.

The polymer particles preferably have on their surface ionic groups and/or groups which are sterically stabilizing in aqueous phase.

Ionic groups are understood here to mean all electrostatically stabilizing groups, in particular anionic and cationic groups.

Sterically stabilizing groups are understood here to mean polymeric or oligomeric chain elements which are at least partially soluble at least in water. They stabilize the dispersed particles on account of entropic effects in accordance with the mechanism of steric stabilization (T. S. Tadros, Interfacial Phenomena and Colloid Stability, Vol. 1 Basic Principles, De Gruyter 2015, 209 ff.) in aqueous media against agglomeration and coagulation. Polymer chains that are generally preferred and suitable as polymeric or oligomeric chain elements are described in the more detailed description of individual polymer particles.

The ionically and/or sterically stabilizing groups are preferably predominantly bonded covalently to the polymer of the particles.

The polymer particles are preferably spherical.

The polymer particles are preferably produced in the form of primary and/or secondary dispersions.

It is additionally preferable for a mean particle size of the polymer particles to be less than about 1 μm, more preferably between 5 to 500 nm, yet more preferably between 5 to 100 nm. The particle size can be determined by dynamic light scattering (e.g. Zeta-Sizer Nano-zs, from Malvern) performed on the aqueous dispersions of the polymer particles. The polymer particles are generally speaking already present after production as aqueous dispersions which are processed for the light scattering measurement by methods known to those skilled in the art. If the polymer particles are present as a dry solid, for example as a powder, they are converted into an aqueous dispersion prior to the light scattering measurement, for example using stirring, dispersing and/or ultrasound devices (e.g. ultrasound homogenizers such as the Sonopuls 4200, from Bandelin).

It is further preferred for the polymer particles to be present as a water-containing dispersion for the production of the water-hardening dental cement, that is to say prior to the hardening of the water-hardening cement.

The dispersed polymer particles of the invention are characterized in particular in that they form a stable disperse phase with water as the dispersion medium. “Stable” here means that the dispersed polymer particles for at least one hour and preferably over a shelf life of a commercial product, preferably at least 6 months, remain substantially dispersed in water as dispersion medium and do not sediment and/or agglomerate and/or aggregate.

It is additionally preferable for a proportion of dispersed polymer particles in the dental cement to be at least 0.005% by weight, more preferably at least 0.01% by weight, based on the overall composition of the dental cement prior to hardening.

It is further preferable for a proportion of dispersed polymer particles in the dental cement to be at most 10% by weight, more preferably at most 3% by weight, yet more preferably at most 1% by weight, further preferably at most 0.5% by weight, most preferably at most 0.3% by weight, based on the overall composition of the dental cement prior to hardening.

It is in addition more preferable for a proportion of dispersed polymer particles in the dental cement to be 0.005% to 5% by weight, more preferably 0.005% to 3% by weight, yet more preferably 0.01% to 1% by weight, further preferably 0.01% to 0.5% by weight, most preferably 0.01% to 0.3% by weight, based on the overall composition of the dental cement prior to hardening.

Preferred polymer particles are polymer particles which have been produced by means of emulsion polymerization.

Preferred polymer particles contain polymer chains which have been formed by optionally substituted homo chains (cf. H. G. Elias, Makromoleküle [Macromolecules], volume 1: Chemische Struktur and Synthesen [Chemical structure and syntheses], 6th edition, Wiley VCH).

Preferred base polymers for the particles are the homopolymers polyacrylates, poly(alkyl)acrylates such as polymethacrylates, polyisoprenes, polybutadienes, polystyrenes, polyvinyl acetates, polyacrylonitriles and polyacrylamides. Particularly preferred base polymers are the copolymers which are polymerized from the monomers of the homopolymers mentioned and/or further monomers. For example, it is particularly preferred to form poly(meth)acrylate particles from two or more acrylate or methacrylate monomers. Further preferred copolymers for use in the polymer particles of the invention are styrene-acrylate copolymers, styrene-maleic acid derivative copolymers, styrene-butadiene copolymers, vinyl acetate-ethylene copolymers, vinyl acetate-vinyl alcohol copolymers, vinyl acetate-acrylate copolymers, acrylonitrile-butadiene copolymers.

It may be advantageous for acidic monomers such as acrylic acid and maleic acid or salts thereof to be used in small amounts for the base polymers mentioned. It may be advantageous for basic monomers such as N,N-dimethylaminoethyl methacrylate or salts thereof to be used in small amounts for the base polymers of the particles. The proportions of the acidic monomers or their salts or of the basic monomers or their salts are preferably below 10% by weight, particularly preferably below 5% by weight.

Particle dispersions of the polymers of the invention having homo chains can be produced particularly advantageously by free-radical emulsion polymerization processes.

To this end, the monomers, optionally with addition of a surfactant, are polymerized in an aqueous phase with a water-soluble, free-radical initiator.

In order to obtain ionically stabilized particle dispersions in which the charge centers are covalently bonded to the surfaces of the particles, in a preferred variant what is called emulsifier-free emulsion polymerization is used [M. Egen, Funktionale dreidimensionale Photonische Kristalle aus Polymerlatizes [Functional three-dimensional photonic crystals made from polymer latices], Dissertation, Johannes Gutenberg University Mainz, 2003]. In this variant of emulsion polymerization, ionic free-radical initiators such as potassium peroxodisulfate, which during the initiating reaction introduce an ionic group into the growing polymer chain, are used. In this way, electrostatically stabilized polymer particles with covalently attached ionic groups are produced even without the use of surfactants.

In a further preferred process for producing polymer particles with covalently attached stabilizing groups, polymerizable surfactants (so-called surfmers) are used, which firstly copolymerize with the monomers in a free-radical reaction and secondly stabilize the dispersion due to their surfactant properties [M. Summers, J. Eastoe, Advances in Colloid and Interface Science, 100-102, (2003), 137-152]. Using surfmers allows both sterically and electrostatically stabilized dispersions to be produced.

Polymer particles based on homo chains may be crosslinked in a specific embodiment.

Crosslinked structures are formed in one production variant by the copolymerization of monomers having a copolymerizable group with monomers having more than one copolymerizable group in the emulsion polymerization process.

Further suitable polymer particles for use in the dental materials of the invention can be produced by miniemulsion polymerization. In this process, suitable particles can be produced both by a free-radical mechanism and by polyaddition (K. Landfester, F. Tiarks, H.-P. Hentze, M. Antonietti, Macromol. Chem. Phys. 201, (2000) 1-5; K. Landfester, Macromol. Rapid Commun. 22, (2001) 896).

Preferred polymer particles contain polymer chains which may have substituted hetero chains. Such polymer particles preferably possess polysiloxane elastomers having slight to substantial crosslinking, preferably the particle core. Preference is given to polydialkyl, polyalkylaryl or polydiarylsiloxane elastomers. The sterically or electrostatically stabilizing groups are preferably attached to the surfaces of the polysiloxane elastomer particles by Si—C bonds.

In one embodiment, the stabilizing groups are poly(meth)acrylic acid copolymer chains. These may be produced, for example, by copolymerization of (meth)acrylic acid-containing monomer mixtures with polysiloxane particles surface-modified by methacrylate groups. Comonomers can be appropriately selected for this process. If, for example, the intention is to increase the negative charge in the copolymer shell, this can be achieved by copolymerization with 0.1%-20% by weight, particularly preferably 0.2%-5% by weight (in each case based on the (meth)acrylic acid) of polymerizable medium-strength or strong acids or salts thereof. Polymerizable sulfonic acids or salts thereof are suitable for this purpose. Derivatives of (meth)acrylamidopropylsulfonic acid or its salts are particularly suitable for this purpose. In the same way, polymerizable acidic phosphoric esters or phosphonic esters, which can be found in the prior art, can be used as comonomers.

In one variant, the suitable polymer particles are producible from polysiloxane particles having a poly(meth)acrylate shell, as are commercially available, for example, under the name Genioperl P52 (Wacker Chemie, Germany), by hydrolysis of the poly(meth)acrylate shell.

Preferred polymer particles are core-shell particles in which an inner portion of the particle differs from an outer portion in terms of material and potentially also in terms of other properties, such as for example the modulus of elasticity. In this context, “shell” is not to be understood as meaning a layer of sterically or electrostatically stabilizing groups. Preferred core-shell particles may have a relatively soft core and a relatively hard shell. In another embodiment, they possess a relatively hard core and a relatively soft shell. The core-shell particles preferably essentially contain homo chains. The core-shell particles are preferably produced via two-stage emulsion polymerization processes. The preferred core-shell particles may be PU particles. In a preferred embodiment, the core-shell particles have a polyurethane core and a shell composed of polymer chains formed by optionally substituted homo chains.

Preferred polymer particles are polyurethane particles (PU particles).

Preference is given to those PU particles produced from:
a) at least one polyisocyanate,
b) optionally at least one compound which is mono- or difunctional in terms of an isocyanate reaction, especially a polyol A,
c) at least one further compound which is mono- or difunctional in terms of an isocyanate reaction and which additionally comprises at least one ionic group and/or at least one functional group which can be converted into an ionic group and/or at least one sterically stabilizing group, and
d) optionally at least one further compound which is mono- or di- or polyfunctional in terms of an isocyanate reaction and is selected from the group of polyol B, chain extender and crosslinker,
   wherein at least one of the two components b) or d) must be present.

Preference is given to those PU particles produced from:
a) at least one polyisocyanate,
b) at least one polyol A,
c) at least one further compound which is mono- or difunctional in terms of an isocyanate reaction and which additionally comprises at least one ionic group and/or at least one functional group which can be converted into an ionic group and/or at least one sterically stabilizing group, and
d) optionally at least one polyol B and/or one chain extender and/or one crosslinker.

Compounds which are mono- or difunctional in terms of an isocyanate reaction are those containing groups which can react with isocyanate. These are preferably functional groups containing active hydrogen, such as for example OH, NH₂, NH, NHNH₂, SH and the like. Such groups are known to those skilled in the art and have been described, for example, in D. Randall, S. Lee (eds.), The Polyurethanes Book, John Wiley & Sons, Ltd, 2002. Those skilled in the art will choose the compounds depending on the desired properties.

Suitable polyisocyanates have at least two isocyanate groups. The at least one polyisocyanate preferably has exactly two isocyanate groups. However, it is also preferable for the at least one polyisocyanate to be a higher-functionality polyisocyanate having more than two isocyanate groups. The at least one polyisocyanate is preferably an aromatic or aliphatic, especially an acyclic or cyclic, compound. It is particularly preferred when the at least one polyisocyanate is an aliphatic, more preferably still a cycloaliphatic, compound. The at least one polyisocyanate is preferably a modified or unmodified. Suitable modified polyisocyanates are for example those containing carbodiimide groups, allophanate groups, isocyanurate groups, urethane groups and/or biuret groups. It is also preferable for a combination of modified and unmodified polyisocyanates to be used.

Examples of preferred polyisocyanates are tetramethylene diisocyanate, hexamethylene diisocyanate, isophorone diisocyanate, 1,4-diisocyanatocyclohexane, 1,3-bis(isocyanatomethyl)cyclohexane, 4,4′-diisocyanatodicyclohexylmethane, 1,4-phenylene diisocyanate, 2,6-toluene diisocyanate, 2,4-toluene diisocyanate, m-xylene diisocyanate, 1,3-bis(1-isocyanato-1-methylethyl)benzene, 2,4′-diphenylmethane diisocyanate and 4,4′-diphenylmethane diisocyanate. Particularly preferred polyisocyanates are selected from aliphatic and cycloaliphatic polyisocyanates.

Polyol A preferably has a hydroxyl group and at least one further hydroxyl or amino group.

In a further embodiment, polyol A has two or more amino groups preferably in a terminal position, such as α,ω-diaminopolyether, for example the Jeffamine® polyetheramine types sold by Huntsman Corporation.

Polyol A preferably has a molecular weight of between 500 and 6000 g/mol, more preferably between 500 and 2000 g/mol. A suitable polyol A may preferably be linear or branched. Examples of suitable polyols A are polyether polyols, polyester polyols, polyesteramide polyols, polycarbonate polyols, polyolefin polyols, polysiloxane polyols and poly(meth)acrylate polyols, more preferably still each with terminal hydroxyl groups. Polyether polyols are particularly preferred. Polytetrahydrofuran 1000 is most preferred.

Polyether polyols are preferably the reaction product of the polymerization of cyclic, organic oxides such as for example ethylene oxide, propylene oxide or tetrahydrofuran by means of polyfunctional initiators such as water, ethylene glycol, propylene glycol, diethylene glycol, cyclohexanedimethanol, glycerol, trimethylolpropane, pentaerythritol. Mixtures of cyclic, organic oxides may also be used. The polymerization of various cyclic, organic oxides can be effected simultaneously to form random copolymers, or the addition can be done successively to form block copolymers.

Polyester polyols are preferably obtained by reacting at least dihydric alcohols with at least dibasic carboxylic acids. These carboxylic acids may be aliphatic, cycloaliphatic, heterocyclic, araliphatic or aromatic. In addition to the free carboxylic acids, corresponding carboxylic anhydrides or the methyl or ethyl esters of the corresponding carboxylic acids may also be used. Mixtures of different polybasic carboxylic acids and derivatives thereof can also be reacted with mixtures of different polyhydric alcohols.

Examples of suitable carboxylic acids, carboxylic anhydrides and carboxylic esters are succinic acid, succinic anhydride, dimethyl succinate, adipic acid, dimethyl adipate, glutaric acid, dimethyl glutarate, cyclohexane-1,4-dicarboxylic acid, maleic acid, maleic anhydride, fumaric acid, phthalic acid, isophthalic acid, phthalic anhydride, tetrahydrophthalic acid, hexahydrophthalic acid or dimethyl terephthalate.

Examples of suitable alcohols are ethylene glycol, diethylene glycol, triethylene glycol, propane-1,2-diol, propane-1,3-diol, dipropylene glycol, butane-1,3-diol, butane-1,4-diol, neopentyl glycol, hexane-1,6-diol, 1,4-bis(hydroxymethyl)cyclohexane, trimethylolpropane, glycerol or pentaerythritol.

Polyester polyols may also be prepared by polymerization of lactones. Examples of suitable lactones are β-propiolactone, γ-butyrolactone or ε-caprolactone.

Suitable polyesteramides are prepared like polyesters, with a portion of the polyhydric alcohols being replaced by amino alcohols, such as for example ethanolamine, or diamines, such as for example ethylenediamine.

Suitable polycarbonate polyols can be obtained by reacting diols such as propane-1,2-diol, propane-1,3-diol, butane-1,4-diol, diethylene glycol with carbonic esters such as diphenyl carbonate, dimethyl carbonate or with phosgene.

Examples of suitable polyolefin polyols are polybutadienes with terminal hydroxyl groups which are sold under the name “Nisso-PB G-Series” from Nippon Soda.

Examples of suitable polysiloxane polyols are polydimethylsiloxanes with terminal hydroxyalkyl groups, preferably hydroxybutyl or hydroxypropyl groups.

Examples of suitable poly(meth)acrylate polyols are copolymers of (meth)acrylic esters with at least one compound having at least one hydroxyl group and at least one (meth)acrylate group. Preference is given to a copolymer of (meth)acrylic esters with at least one compound with only one hydroxyl group and only one (meth)acrylate group.

The additionally at least one ionic group, of the at least one further compound which is mono- or difunctional in terms of an isocyanate reaction, is preferably an anionic or cationic group.

The anionic group is preferably a carboxylate, sulfonate, sulfate, sulfonium, phosphate or phosphonate group. The cationic group is preferably an ammonium or phosphonium group. The ionic groups and/or the functional group which can be converted into an ionic group can be incorporated both into the PU main chain and laterally onto the PU main chain.

Sterically stabilizing groups are polymeric or oligomeric chain elements which are at least partially soluble at least in water and are preferably covalently attached to the polyurethane particles. They stabilize the particles on account of entropic effects in accordance with the mechanism of steric stabilization (T. S. Tadros, Interfacial Phenomena and Colloid Stability, Vol. 1 Basic Principles, De Gruyter 2015, 209 ff.) in aqueous media against agglomeration and coagulation.

Examples of suitable polymer chains here are water-soluble polymer chains such as poly(oxyethylene), poly(oxazolines), water-soluble poly(2-alkyloxazolines), further water-soluble poly(ethyleneimine) derivatives, poly(N-vinylpyrrolidones), water-soluble poly((meth)acrylates), such as for example poly(hydroxyethyl (meth)acrylate), poly((meth)acrylamides), water-soluble polysaccharides such as for example starches, pectins, cellulose and cellulose ethers such as methyl cellulose or hydroxyethyl cellulose, proteins such as for example gelatins, partially hydrolyzed polyvinyl acetate or polyvinyl alcohol.

In a preferred embodiment, the polymer chains do not bear any groups that are charged between a pH of 1 and 9 in water.

In a further embodiment, the polymer chains bear additional groups which bear a charge between a pH of 1 and 9. These may be positive or negative charge carriers. Suitable chemical groups are the groups described as ionic groups.

In a specific embodiment, the sterically stabilizing group which also bears charges is a poly(meth)acrylic acid chain or a poly(meth)acrylic acid copolymer chain.

Suitable relative molecular weights of the sterically stabilizing polymer chains are between 200 and 100 000, preferably between 300 and 20 000 and particularly preferably between 400 and 4000.

In one embodiment, sterically stabilizing groups are polymer chains which have been covalently bonded to the particle by in each case a chain segment. This chain segment can be located at the chain end or at another position of the chain. The connecting chain segment is preferably located at the chain end.

In a further preferred embodiment, the additionally at least one ionic and/or sterically stabilizing group, of the at least one further compound which is mono- or difunctional in terms of an isocyanate reaction, is a sterically stabilizing chain.

The sterically stabilizing chain can be attached to the particle in various ways. In a preferred embodiment, the chain is attached to the particle via a urethane group or a urea group.

Typical sterically stabilizing groups which are joined to the particle via a chain segment are terminally monofunctional methoxypoly(oxyethylene), which is for example introduced during the particle synthesis in the form of α-methoxy,ω-hydroxypoly(oxyethylene) or α-methoxy,ω-aminopoly(oxyethylene), or poly(methyloxazoline), which is for example introduced during the particle synthesis in the form of terminally monohydroxy- or amino-functionalized poly(methyloxazoline). They may also be attached laterally to polyurethane chains.

Sterically stabilizing groups in a further embodiment are polymer chains which are covalently bonded to the particle via two or more chain segments.

Preference is given to polymer chains which are bonded to the particle via two chain segments. In one preferred embodiment, the bonding segments are located at the chain ends. It may be preferable here for the sterically stabilizing polymer chains to be introduced during the synthesis of the particle in the form of polyol A or polyol B. In a preferred embodiment, the chains are attached to the particle via a urethane group or a urea group.

A typical example of a sterically stabilizing group which is bonded to the particle via two chain segments is poly(oxyethylene), which is introduced for example during the particle synthesis in the form of polyethylene glycol (polyol A or B) or else α,ω-diaminopoly(oxyethylene). Another example is hydroxy- or amino-telechelic poly(methyloxazoline) or poly(ethyloxazoline).

The at least one further compound which is mono- or difunctional in terms of an isocyanate reaction is particularly preferably hydroquinone monosulfonic acid potassium salt, N-methyldiethanolamine, PEG350, PEG600, TEGOMER® D3403 or Ymer® N 120.

The at least one polyol B is preferably a polyol B having at least two hydroxyl groups. The at least one polyol B preferably has a molecular weight of less than 500 g/mol. A suitable polyol B may preferably be aromatic, especially carbocyclic or heterocyclic, or aliphatic, especially linear, branched or cyclic. The polyol B is particularly preferably aliphatic. Polyols B are preferably at least dihydric alcohols. Examples of suitable polyols B are ethylene glycol, diethylene glycol, triethylene glycol, propane-1,2-diol, propane-1,3-diol, dipropylene glycol, butane-1,3-diol, butane-1,4-diol, neopentyl glycol, hexane-1,6-diol, 1,4-bis(hydroxymethyl)cyclohexane, trimethylolpropane, glycerol and pentaerythritol.

The chain extender is preferably a diamine having a molecular weight of less than 500 g/mol. Preferred diamines are α,ω-alkylenediamines such as ethylenediamine, 1,3-propylenediamine, 1,4-butylenediamine, 1,6-hexamethylenediamine or else higher molecular weight primary and secondary diamines, particularly preferably ethylenediamine. Further preferred embodiments of diamines are diamines the organic radical (which lies between the amino groups) of which bears further heteroatoms, especially oxygen atoms.

The crosslinker is preferably one having at least three functional groups, such as for example diethylenetriamine.

The production of such polyurethane particles has been described, inter alia, in:
Robin et al., Polym. Int. 61, 495-510, 2012,
Ramesh et al., J. Macromol. Sci. C, 38, 481-509, 1998,
Long et al., Macromol. Chem. Phys. 215, 2161-2174, 2014, and
Kim, Colloid Polym. Sci., 274, 599-611, 1996.

The PU particles of the invention bear, in each case depending on the selected component b), c) and/or d), urethane, urea, allophanate and/or biuret groups as linkage sites. All of these groups may be present, or just particular ones. It may be preferable to use PU particles containing exclusively urethane groups as linkage sites from the polyaddition step. For reasons of hydrolysis stability, it may also be preferable to use PU particles exclusively or at least predominantly containing urea groups as linkage sites from the polyaddition step.

It is preferable for the polymer particles not to have any polymerizable groups, in particular not to have any (alkyl)acrylate or (alkyl)acrylamide groups.

### Acid-Reactive Powder

Suitable acid-reactive powders are known to those skilled in the art. It is preferable for the acid-reactive powder to be selected from metal oxides, metal hydroxides, mineral trioxide aggregate (MTA), hydroxyapatite, bioactive glasses, acid-reactive glasses and mixtures thereof.

The metal oxides are preferably selected from magnesium oxide, calcium oxide, strontium oxide, barium oxide, zinc oxide, lanthanum oxide, yttrium oxide and mixtures of these.

The metal hydroxides are preferably selected from magnesium hydroxide, calcium hydroxide, strontium hydroxide, lanthanum hydroxide, yttrium hydroxide and mixtures of these.

Mineral trioxide aggregate (MTA) is a powder consisting of the main components calcium silicate (dicalcium silicate ((CaO)₂.SiO₂) and tricalcium silicate ((CaO)₃.SiO₂)), tricalcium aluminate ((CaO)₃.Al₂O₃) and calcium oxide. Further constituents present may preferably be gypsum (CaSO₄.2H₂O) and bismuth(III) oxide.

Bioactive glasses are a group of surface-active glasses which have bioactivity. They are distinguished, in contrast to conventional glasses, by the fact that they are soluble in an aqueous medium and form a hydroxyapatite layer on their surface. Examples of preferred bioactive glasses are Bioglass 45S5 or bioglasses as are described in WO 2011/000866, WO 2011/161422 and WO 2014/154874.

Acid-reactive glasses used may preferably be aluminosilicate glasses, more preferably fluoroaluminosilicate glasses, yet more preferably calcium fluoroaluminosilicate glasses and strontium fluoroaluminosilicate glasses. In addition, glasses containing lanthanides are also furthermore preferred. Suitable glasses are in particular those as described for example in EP 0 885 854 B1, DE 3804469 C2 or EP 1343452 B1.

Preferred constituents of suitable glass powders are SiO₂, Al₂O₃, CaF₂, AlF₃, NaF, AlPO₄, CaO, SrO, SrF₂, P₂O₅, B₂O₃, Bi₂O₃, MgO, TiO₂, ZrO₂, GeO₂, La₂O₃ or further oxides of the lanthanide series and ZnO. Suitable combinations are for example those described in A. D. Wilson and J. W. Nicholson, Acid-base cements: Their biomedical and industrial applications, Cambridge Press, 1993.

It may be preferable for the surfaces of a powder not to have been treated or modified with organic compounds. It is even more preferable for them not to comprise any polymerizable groups, in particular not to comprise any (alkyl)acrylate or (alkyl) acrylamide groups.

The acid-reactive powders preferably have mean particle sizes (d50) of from 0.5 to 30 μm, more preferably 1 to 20 μm and/or a maximum particle size (d99) of <150 μm, preferably <100 μm, more preferably <80 μm (e.g. Laser Particle Sizer, Beckman Coulter LS 13320). The acid-reactive powder preferably comprises a first quantity of glass particles having a mean particle size of from 5 to 20 μm, more preferably 5 to 15 μm, and a second quantity of glass particles having a mean particle size of from 1 to 5 μm, more preferably 2 to 3 μm. The first and second quantity of the glass particles differ from each other, by way of example by a different glass composition, a surface treatment conducted and/or a different shape, such as for example an irregular or round form.

A proportion of acid-reactive powder in the dental cement is preferably 20% to 90% by weight, more preferably 40% to 85% by weight, based on the overall composition of the dental cement prior to hardening.

### Polyprotic Acid

Suitable polyprotic acids are known to those skilled in the art. It is preferable for the polyprotic acid to be selected from polyacids and phosphoric acid, particularly preferably polyacid.

Preferred polyacids are homopolymers and copolymers of unsaturated carboxylic acids or phosphonic acids. More preference still is given to polyalkenoic acids as polyacids. Particular preference is given to polyacrylic acid (PAA), poly(acrylic acid-co-maleic acid), poly(acrylic acid-co-itaconic acid), poly(acrylic acid-co-vinylphosphonic acid) and poly(vinylphosphonic acid). Further monomers and comonomers for suitable homopolymers and copolymers are described in Schricker et al., J. Mat. Chem. 22, 2824-2833, 2012, such as for example N-vinylcaprolactam, N-vinylpyrrolidone, amino acid-modified acrylates and acrylamides.

In a preferred embodiment, the polyprotic acid does not contain any free-radically polymerizable group, in particular does not contain any (alkyl)acrylic ester or (alkyl)acrylamide group.

A proportion of polyprotic acid in the dental cement is preferably 4.9% to 40% by weight, more preferably 7.4% to 25% by weight, based on the overall composition of the dental cement prior to hardening.

### Water

A proportion of water in the dental cement is preferably 4.9% to 40% by weight, more preferably 7.4% to 25% by weight, based on the overall composition of the dental cement prior to hardening.

### Further Additional Constituents

In a preferred embodiment, the water-hardening dental cement comprises further additional constituents selected from the group consisting of complexing agents, inorganic and organic fillers, inorganic and organic colorants and mixtures of these. Suitable complexing agents are for example tartaric acid, citric acid and/or those described in Prosser et al., J. Dent. Res., 61, 1982, 1195-1198. Suitable inorganic fillers are for example X-ray opaque non-acid-reactive (inert) inorganic fillers.

The invention further provides a method for producing a water-hardening dental cement, wherein at least an acid-reactive powder, a polyprotic acid, water and dispersed polymer particles are mixed.

The method of the invention for producing a water-hardening dental cement can be developed with further features which are described in connection with the water-hardening dental cement of the invention and the kit of the invention.

The invention further provides a kit for producing a water-hardening dental cement comprising the constituents:
a) dispersed polymer particles,
b) an acid-reactive powder,
c) a polyprotic acid, and
d) water.

It is preferable for a proportion of dispersed polymer particles in the kit to be at least 0.005% by weight, more preferably at least 0.01% by weight, based on the overall composition of the kit prior to hardening of the dental cement. The weight figures relate to the polymer particles as such and not to the particle dispersion.

It is further preferable for a proportion of dispersed polymer particles in the dental cement to be at most 10% by weight, more preferably at most 3% by weight, yet more preferably at most 1% by weight, further preferably at most 0.5% by weight, most preferably at most 0.3% by weight, based on the overall composition of the kit prior to hardening of the dental cement.

It is in addition more preferable for a proportion of dispersed polymer particles in the dental cement to be 0.005% to 5% by weight, more preferably 0.005% to 3% by weight, yet more preferably 0.01% to 1% by weight, further preferably 0.01% to 0.5% by weight, most preferably 0.01% to 0.3% by weight, based on the overall composition of the kit prior to hardening of the dental cement.

A proportion of acid-reactive powder in the kit is preferably 20% to 90% by weight, preferably 40% to 85% by weight, based on the overall composition of the kit prior to hardening of the dental cement.

A proportion of polyprotic acid in the kit is preferably 4.9% to 40% by weight, more preferably 7.4% to 25% by weight, based on the overall composition of the kit prior to hardening of the dental cement.

A proportion of water in the kit is preferably 4.9% to 40% by weight, more preferably 7.4% to 25% by weight, based on the overall composition of the kit prior to hardening of the dental cement.

The kit of the invention can be developed with further features which are described in connection with the water-hardening dental cement of the invention, especially in connection with the further and additional constituents thereof.

It is preferable for the kit to be composed of at least two components and for the constituents of the kit to be divided between these components. The constituents of the kit may, depending on the embodiment, preferably be present in the first and/or second component, as long as the constituents made up of acid-reactive powder, polyprotic acid and water are not present in just one single component. Different combinations are associated with respectively different advantages in this case, for example in terms of storage stability and miscibility. The dispersed polymer particles may, depending on the embodiment, preferably be present in the first and/or second component. In a preferred embodiment, the first component contains the acid-reactive powder and the second component contains water and the dispersed polymer particles. Depending on the embodiment, the polyprotic acid is present in the first and/or second component. It is necessary that the at least two components are mixed shortly before use for the production of the water-hardening dental cement.

It is further preferable for a first component of the kit to be provided as a powder and a second component to be provided as a liquid, or for a first component of the kit to be provided as a paste and a second component to be provided as a paste.

It is preferable in addition for the kit to have apparatuses suitable for mixing the components. Preferred apparatuses for mixing are for example spatulas and mixing pads and/or apparatuses in which the parts of the kit are present in pre-metered form and/or apparatuses for automatic mixing of the parts.

A mixing ratio of a powder component to a liquid component is preferably greater than 1:1, more preferably greater that 2:1, yet more preferably greater than 3:1. This means that, for example, 3.5 parts by weight of powder are mixed with 1 part by weight of liquid.

Examples of suitable kit component systems that may be mentioned include the capsules described in EP 1 344 500 B1, and cartridge systems for automatic mixing, such as for example the commercially available Mixpac™ L and S systems. The invention further provides for the use of the water-hardening dental cement of the invention as a filling, yet more preferably as a masticatory load-bearing filling.

The invention will now be explained using advantageous embodiments with reference to the attached drawings. In the figures:

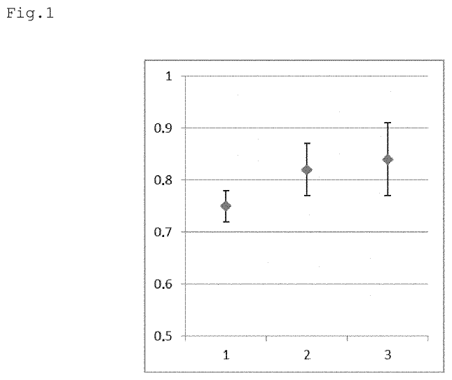

FIG. 1: Average values and standard deviations for the fracture toughness (stress intensity factor K_{Ic} (in MPa·\sqrt{m})) of the cements 1-3 shown in table 4.

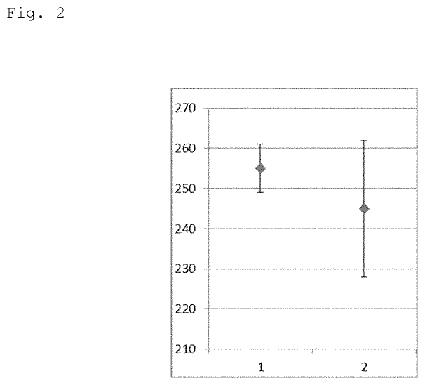

FIG. 2: Average values and standard deviations for the compressive strength (in MPa) of the cements 1-2 shown in table 4.

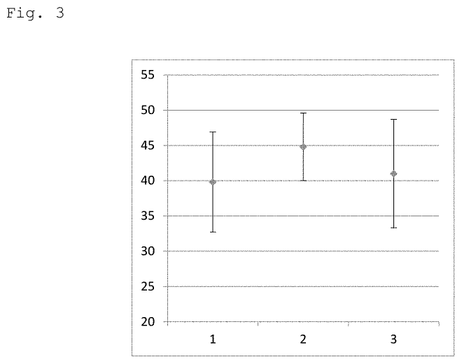

FIG. 3: Average values and standard deviations for the flexural strength (in MPa) of the cements 1-3 shown in table 4.

### CHEMICALS AND THE PRETREATMENT/USE THEREOF

| | |
|---|---|
| Polytetrahydrofuran 1000 | Merck KGaA |
| 4,4′- | Sigma Aldrich |
| Diisocyanatodicyclohexylmethane | |
| (H12MDI) | |
| Hydroquinone monosulfonic acid | Sigma Aldrich |
| potassium salt | |
| N-Methyldiethanolamine | Sigma Aldrich |
| n-Butyl methacrylate | Sigma Aldrich |
| Potassium peroxodisulfate | Sigma Aldrich |
| Methyl methacrylate | Evonik Performance |
| | Materials GmbH |
| Polyacrylic acid, M_{N} = | |
| 14 000 g/mol, M_{W} = | |
| 49 000 g/mol, ground, d50 less | |
| than 50 μm | |
| Hyperpure water (GPR Rectapur) | VWR International |

The polytetrahydrofuran 1000 was heated to 60° C. for 4 hours at 0.03 mbar so that a dried polytetrahydrofuran (PTHF) was obtained. The powder was stored under nitrogen.

### Fluoroaluminosilicate Glass A (FAS A):

| | | |
|---|---|---|
| Composition | Si as SiO₂ | 32.2% by weight |
| | Al as Al₂O₃ | 31.6% by weight |
| | Sr as SrO | 24.9% by weight |
| | P as P₂O₅ | 5.2% by weight |
| | Na as Na₂O | 1.7% by weight |
| | F as F⁻ | 7.2% by weight |

The glass powder was ground in a ball mill to a mean particle diameter d₅₀=2.6 μm. The powder was heat treated for 8 hours at 500° C.

### Fluoroaluminosilicate Glass B (FAS B):

| | | |
|---|---|---|
| Composition | SiO₂ | 36.00% by weight |
| | Al₂O₃ | 22.50% by weight |
| | CaF₂ | 21.00% by weight |
| | Na₃AlF₆ | 9.00% by weight |
| | AlF₃ | 6.60% by weight |
| | AlPO₄ | 5.00% by weight |

The glass powder was ground in a ball mill to a mean particle diameter d₅₀=7.7 μm. 1 kg of the powder was subsequently suspended in a solution of 30 g of KH₂PO₄ in 3 l of distilled water and stirred for 24 hours at room temperature (RT). The suspension was then filtered, washed with distilled water and dried for 6 hours at 100° C.

### Methods

Mean particle diameter and average zeta potential of the ionic polyurethane particles (PU particles):

The parameters were determined by means of dynamic light scattering using a ‘Zetasizer Nano-ZS’ from Malvern. The mean particle diameter was measured as the average hydrodynamic equivalent radius in the form of the Z-average. The particles were in the form of aqueous dispersions. Dispersions after production were 1:10 diluted with hyperpure water. For the measurements, water was used as dispersing medium with the following parameters:

refractive index 1.33,
dielectric constant 78.5, and
viscosity 0.8873 cP.

The measurements were conducted at 25° C.

Particle size of the fluoroaluminosilicate glasses:

The particle size distribution and the mean particle diameter (d₅₀) were determined with a Beckman Coulter Laser Particle Sizer LS130 and a Beckman Coulter Laser Particle Sizer LS13320.

250 mg of the ground glass was mixed with 4 drops of glycerol on a roughened watch glass to give a creamy paste. This paste was predispersed with 1 drop of water using a pestle. The paste was subsequently mixed into 5 ml of water and dispersed in an ultrasonic bath (Bandelin Sonorex RK102H) for 5 minutes with ice-water cooling. The dispersion was introduced into the measurement chamber of the particle sizer (Coulter LS130 or Beckman-Coulter LS13320) and measured while circulating the aqueous dispersion to be measured.

The measurement was effected in tap water. The evaluation was effected according to the Fraunhofer diffraction optical model.

### Freeze Drying:

Diluted solutions were freeze-dried using the ‘Sublimator VaCo 5’ freeze drier from ZIRBUS technology GmbH.

### Flexural Strength (FS):

The flexural strength was measured in accordance with ISO 9917-2:2010 at an advance rate of 0.8 mm/min.

Compressive Strength (CS):

The compressive strength was measured in accordance with ISO 9917-1:2010 at an advance rate of 1 mm/min.

### Fracture Toughness (K_{IC}):

Powder and liquid were mixed in the respectively specified mixing ratio within 1 minute using a spatula on a pad and filled into a mold having the dimensions length L=50 mm, height H=4 mm and width W=3 mm. After 1 hour at 37° C. and >95 relative humidity, the test specimen was demolded and stored for a further 23 hours±1 hour in distilled water at 37° C. The test specimens were notched on one of the 3 mm-wide sides with a low-speed saw (Isomet from Buehler; diamond cutting disk D46/54, thickness 0.20 mm, from Boma). The notch depth was approx. 0.7 mm.

The width and the height of a test specimen were measured using a caliper. The test specimen was placed with the notch facing downwards on the 3-point bending fracture bending apparatus (spacing between the supports S=20 mm) of a universal testing machine (Zwick Z2.5, from Zwick Roell), so that the notch was located exactly underneath the force-transmitting wedge. The measurement of the maximum force (Fₘₐₓ) until fracture of the test specimens was conducted at an advance rate of 0.8 mm/min.

An image of the cross-sectional area of the broken test specimen was recorded under a microscope (Leica Leitz DMRX) using a digital camera (Leica DFC295). The notch depths a1, a2 and a3 of the notch were ascertained at three locations using evaluation software (Leica Application Suite V3). The mean value a was formed from the three values in accordance with the formula. The measured values were used to calculate the fracture toughness (stress intensity factor K_{IC}) according to the formula specified.

$$a = \frac{a 1 + a 2 + a 3}{3}$$

$$f ( \frac{a}{H} ) = \frac{3 {( \frac{a}{H} )}^{1 / 2} [ 1.99 - ( \frac{a}{H} ) ( 1 - ( \frac{a}{H} ) ) \{ 2.15 - 3.93 ( \frac{a}{H} ) + 2.7 {( \frac{a}{H} )}^{2} \} ]}{2 ( 1 + 2 ( \frac{a}{H} ) ) {( 1 - ( \frac{a}{H} ) )}^{3 / 2}}$$

$${K}_{IC} = \frac{{F}_{max} \cdot S \cdot {10}^{- 6}}{B \cdot {H}^{3 / 2}} \cdot f ( \frac{a}{H} )$$

### Example 1

Synthesis of the PU Particles with Anionic Groups:

10 g of PTHF, 13.9 ml of acetone, 5.25 g of H12MDI and 0.02 ml of catalyst solution (dimethyltin dineodecanoate in toluene, proportion by mass 50%) were heated to 60° C. for 4 hours (reflux condenser, drying tube) and then cooled down to room temperature (RT). The isocyanate group content was determined by means of titration. It was 2.96% by weight.

At room temperature (approx. 23° C.), 1.65 g of hydroquinone monosulfonic acid potassium salt (DMSO solution, proportion by mass 10%) were then added, and the mixture was heated to 60° C. for 4 hours and subsequently heated to 70° C. for 1.5 hours, and then cooled down to RT. The isocyanate group content was 0.43% by weight.

Next, the mixture was heated to 50° C. and 38.3 ml of deionized water were added dropwise over approx. 35 min. A milky-white cloudiness developed. The acetone was removed on a rotary evaporator. The mean particle diameter of the remaining dispersion was Z-average=77 nm and the average zeta potential=−31 mV.

The dispersion was purified by means of dialysis. To this end, the dispersion containing approx. 20% by weight of polyurethane particles was diluted with deionized water. 100 ml of the diluted dispersion were dialyzed over 5 days against 8 liters of deionized water (dialysis tube made from regenerated cellulose (‘Zellutrans’, Carl Roth GmbH, MWCO=6000-8000)). The water was changed four times during this time. The solids content of the dispersion was ascertained by freeze drying and was 0.97% by weight.

### Example 2

Synthesis of the PU Particles with Cationic Groups:

10 g of PTHF, 13.9 ml of acetone, 5.25 g of H12MDI and 0.02 ml of catalyst solution were heated to 60° C. for 4 hours and then cooled down to RT. The isocyanate group content was 3.25% by weight.

0.956 ml of N-methyldiethanolamine were added, and the mixture was heated to 60° C. for a further 4 hours and cooled down again.

Next, 1.94 ml of glacial acetic acid and 47 ml of acetone were added, the mixture was heated to 40° C. and then 35 ml of deionized water were added dropwise over approx. 35 min. A milky-white cloudiness developed.

The acetone was removed on a rotary evaporator. The mean particle diameter of the remaining transparent dispersion was Z-average=35 nm and the average zeta potential was 69 mV. The solids content was 22.2% by weight.

### Example 3

Synthesis of the PU particles without ionic groups:

### Batch 1:

| H12MDI | 0.04 | mol |
|---|---|---|
| Butane-1,4-diol | 0.005 | mol |
| Terathane 650 | 0.005 | mol |
| PEG600 | 0.01 | mol |
| DABCO | 1 spatula tip | |
| DBTDL | 3 | drops |

| | |
|---|---|
| S | acetone |

### Batch 2:

| H12MDI | 0.2 | mol |
|---|---|---|
| PolyTHF250 | 0.1 | mol |
| PEG350 | 0.1 | mol |
| DABCO | 1 spatula tip | |
| DBTDL | 3 | drops |

| | |
|---|---|
| S | THF |

Diisocyanate and 2 diol components were each dissolved in the specified solvent (S). In addition, 1,4-diazabicyclo[2.2.2]octane (DABCO) and dibutyltin dilaurate (DBTDL) as catalyst were added to the reaction solution. The solution was stirred for 24 hours, so that a complete reaction was achieved. The prepolymers obtained were added dropwise to an excess of water with vigorous stirring. Particles formed instantaneously in the process. The aqueous particle suspensions obtained were purified three times with water by means of ultrafiltration. The residue from the ultrafiltration was in each case redispersed with a few milliliters of water. The resulting dispersions were analyzed gravimetrically for their solids content and subsequently used to produce the liquids for the glass ionomer cements. The size of the particles obtained was determined using the particle sizer:

### Batch 1

Solids content of the dispersion: 18.6% by weight

Size of the particles: 300 nm

### Batch 2

Solids content of the dispersion: 16.3% by weight

Size of the particles: 165 nm

### Example 4

### Production of the Kit Constituents:

### Powder:

18.2 parts of polyacrylic acid (PAA) and 81.8 parts of FAS B were mixed together.

### Liquid:

The liquids of the invention were prepared by mixing deionized water and the dispersions obtained in example 3.

### Production of Water-Hardening Dental Cements:

Powder and liquid were mixed in the weight ratios given in tables 1 and 2, test specimens were formed and the fracture toughness was determined. The fracture toughness K_{1c} and the standard deviation (SD) are given.

TABLE 1
Production of the water-hardening cements using the
dispersion (Disp) from batch 1, example 3 (300 nm).

| | | | PU | | | |
|---|---|---|---|---|---|---|
| | | | particles | | | |
| | | Liquid | in the | | | |
| | | composition | mixture | K_{1c} | | |
| Powder | Liquid | m(H₂O):m(Disp) | [% by | [MPa/ | | Increase |
| [g] | [g] | [g/g] | weight] | m^{1/2}] | SD | [%] |
| 5.40 | 1.00 | 1.00:0 | 0 | 0.365 | 0.038 | — |
| 1.40 | 0.268 | 1.44:0.276 | 0.48 | 0.430 | 0.075 | 18 |
| 5.40 | 1.19 | 0:1.00 | 3.35 | 0.461 | 0.039 | 26 |

TABLE 2
Production of the water-hardening dental cements using
the dispersion from batch 2, example 3 (165 nm).

| | | | PU | | | |
|---|---|---|---|---|---|---|
| | | Liquid | particle | | | |
| | | composition | mixture | K_{1c} | | |
| Powder | Liquid | m(H₂O):m(Disp) | [% by | [MPa/ | | Increase |
| [g] | [g] | [g/g] | weight] | m^{1/2}] | SD | [%] |
| 5.40 | 1.00 | 1.00:0 | 0 | 0.365 | 0.038 | — |
| 5.40 | 1.03 | 3.78:3.78 | 1.31 | 0.465 | 0.037 | 27 |

### Example 5

### Production of the Kit Constituents (Powder and Liquid)

### Powder:

47.5 parts of FAS A, 31.7 parts of FAS B and 20.8 parts of PAA were mixed together.

### Liquid:

The liquids were prepared by mixing deionized water, tartaric acid and the dispersions obtained in examples 1 and 2. The composition of the liquids is given in table 3.

TABLE 3
Composition of the liquids.

| | Liquid 1 (L1) | | Liquid 2 (L2) | | Liquid 3 (L3) | |
|---|---|---|---|---|---|---|
| | [g] | [%] | [g] | [%] | [g] | [%] |
| Water | 23.78 | 94.97 | 5.366 | 50.95 | 18.435 | 92.11 |
| Dispersion | — | — | 4.640 | 44.06 | — | — |
| from example 1 | | | | | | |
| Dispersion | — | — | — | — | 0.575 | 2.87 |
| from example 2 | | | | | | |
| Tartaric acid | 1.26 | 5.03 | 0.526 | 4.99 | 1.004 | 5.02 |
| Sum total | 25.04 | 100.00 | 10.532 | 100.00 | 20.014 | 100.00 |
| PU content | — | — | 0.045 | 0.427 | 0.128 | 0.640 |

### Example 6

### Production of Water-Hardening Dental Cements:

2.4 g of powder and 0.38 g of liquid were in each case mixed on a pad within a minute using a spatula and test specimens were formed. The compressive strength (CS), flexural strength (FS) and fracture toughness (K_{IC}) of the set test specimens were determined as described.

TABLE 4
Properties of the water-hardening dental cements.

| | Cement 1 | Cement 2 | Cement 3 |
|---|---|---|---|
| Powder [g] | 2.4 | 2.4 | 2.4 |
| L1 [g] | 0.38 | — | — |
| L2 [g] | — | 0.38 | — |
| L3 [g] | — | — | 0.38 |
| CS (MPa, 24 h) | 255 ± 6 | 245 ± 17 | — |
| FS (MPa, 24 h) | 39.8 ± 7.1 | 44.8 ± 4.8 | 41.0 ± 7.7 |
| K_{IC} (MPa\sqrt{m}, 24 h) | 0.75 ± 0.03 | 0.82 ± 0.05 | 0.84 ± 0.07 |

The proportions by mass of ionic PU particles in the water-hardening dental cements were 0.06% and 0.09% by weight. It can be seen that the addition of small amounts of these polyurethane particles brought about a marked increase in the fracture toughness (stress intensity factor K_{IC} of the cements (cf. FIG. 1), while the addition had no effect on compressive strength and flexural strength (cf. FIGS. 2 and 3).

### Example 7—Synthesis of PMMA Particles

A 250 ml three-neck flask equipped with a precision glass stirrer and two septa was initially charged with 150 ml of ultrapure water. The contents were heated to 90° C. under a stream of nitrogen. After 45 min, the nitrogen stream was switched off and 15 ml (141 mmol) of methyl methacrylate were added through the septum. In order to initiate the polymerization, after a further 30 min at 90° C. 5 ml (1.8 mmol) of a 10% aqueous potassium peroxodisulfate solution were added as initiator. This solution had likewise been flushed beforehand with nitrogen for 10 min at 90° C. The reaction solution was stirred at 400 rpm with the precision glass stirrer. For monitoring the reaction, every 30 min 0.1 ml of reaction solution was withdrawn through the septum and dried in air on a glass substrate. When the reflection color of the dried film no longer changed, the solution was stirred for a further 30 min at 90° C. In order to end the reaction, the septum was removed and the suspension was stirred in air for approximately a further 20 min.

The reaction solution was filtered off warm for purification, in order to remove coarse impurities. The filtrate was subsequently centrifuged. At the start, centrifugation was performed at least twice for 5 to 10 min at 4000 rpm, in order to remove colorless sediment. Thereafter, the solution was centrifuged for 30 to 90 min, until a clear solution had formed above the iridescent sediment. The liquid phase was decanted and the sediment redispersed again in 60 ml of distilled water. This procedure was repeated three to four times in order to fully clear the polymer of low molecular weight reaction residues.

Storage was effected as a 5% to 20% aqueous suspension. The size of the particles obtained was determined using a particle sizer from Beckmann-Coulter with a mean particle size of 342 nm.

### Example 8—Synthesis of PMMA-co-n-butylMA Particles (80:20)

Synthesis was effected analogously to example 7—Synthesis of the PMMA particles. However, 12 ml (113 mmol) of methyl methacrylate and 4.5 ml (28 mmol) of n-butyl methacrylate were added.

The size of the particles obtained was determined with a mean particle size of 323 nm.

### Example 9—Synthesis of PMMA-co-n-butylMA Particles (60:40)

Synthesis was effected analogously to example 7—Synthesis of the PMMA particles. However, 9 ml (84.6 mmol) of methyl methacrylate and 9 ml (56 mmol) of n-butyl methacrylate were added.

The size of the particles obtained was determined with a mean particle size of 345 nm.

### Example 10—Synthesis of PMMA-co-n-butylMA Particles (40:60)

Synthesis was effected analogously to example 7—Synthesis of the PMMA particles. However, 6 ml (56 mmol) of methyl methacrylate and 13.6 ml (85 mmol) of n-butyl methacrylate were added.

The size of the particles obtained was determined with a mean particle size of 332 nm.

### Example 11—Synthesis of Core-Shell Particles (PnbutylMA-PMMA)

Synthesis was initially effected analogously to example 7—Synthesis of the PMMA particles. However, 13.6 ml (85 mmol) of undistilled n-butyl methyl methacrylate were first added. When the reflection color of the dried film no longer changed, 6 ml (56 mmol) of methyl methacrylate were added through the septum and the reaction was then continued again analogously to example 7.

The size of the particles obtained was determined with a mean particle size of 380 nm.

### Example 12

### Kit Constituents

The powder consisted of a homogeneous mixture of fluoroaluminosilicate glass B (FAS B) and polyacrylic acid in the ratio 4.51:1.

The liquid consisted of demineralized water for the reference system or an aqueous particle dispersion for a particle-reinforced glass ionomer cement. The particle dispersions were each produced by redispersing an appropriate amount of polymer particles in demineralized water. The individual particle contents of the aqueous particle dispersions are given in table 5.

### Example 13

### Production of Glass Ionomer Cements

The glass ionomer cements were mixed from the kit constituents using a spatula on a pad. The individual mixing ratios are given in table 5.

TABLE 5

| | | | | Polymer | | | |
|---|---|---|---|---|---|---|---|
| | | | Polymer | particles | | | |
| | | | particles | in the | | | |
| | | | in the | cement | | | |
| Polymer | Powder | Liquid | liquid | [% by | K1c | | Increase in |
| particles | [g] | [g] | [g/g] | weight] | [MPa/m^{1/2}] | SD | K1c [%] |
| Reference | 1.4 | 0.26 | 0 | 0 | 0.365 | 0.037 | — |
| Example 7 | 1.4 | 0.264 | 0.015 | 0.25 | 0.402 | 0.026 | 10.06 |
| | 1.4 | 0.268 | 0.03 | 0.48 | 0.412 | 0.045 | 12.99 |
| | 1.4 | 0.273 | 0.05 | 0.75 | 0.406 | 0.042 | 10.06 |
| Example 8 | 1.4 | 0.268 | 0.03 | 0.48 | 0.388 | 0.049 | 6.29 |
| Example 9 | 1.4 | 0.268 | 0.03 | 0.48 | 0.400 | 0.041 | 9.64 |
| Example 10 | 1.4 | 0.268 | 0.03 | 0.5 | 0.425 | 0.025 | 16.35 |
| Example 11 | 1.4 | 0.264 | 0.015 | 0.25 | 0.400 | 0.044 | 9.64 |
| | 1.4 | 0.268 | 0.03 | 0.5 | 0.402 | 0.033 | 10.27 |
| | 1.4 | 0.277 | 0.065 | 1 | 0.410 | 0.025 | 12.37 |

As a result of the addition of 0.25% to 0.75% by weight of the polymer particles from examples 7 to 11 in aqueous dispersion, the fracture toughness of the glass ionomer cement (reference) was markedly improved.

1. A water-hardening dental cement comprising an acid-reactive powder, a polyprotic acid, water and dispersed polymer particles.

2. The water-hardening dental cement as claimed in claim 1, characterized in that the water-hardening dental cement is a glass ionomer cement, more preferably a conventional glass ionomer cement.

3. The water-hardening dental cement as claimed in claim 1, characterized in that a mean particle size of the polymer particles is less than about 1 μm, more preferably between 5 to 500 nm, yet more preferably between 5 to 100 nm, determined by dynamic light scattering in aqueous dispersion (in water).

4. The water-hardening dental cement as claimed in claim 1, characterized in that the polymer particles have been dispersed in aqueous solution prior to the hardening of the water-hardening cement.

5. The water-hardening dental cement as claimed in claim 1, characterized in that a proportion of dispersed polymer particles in the cement is 0.005% to 10% by weight, more preferably 0.005% to 3% by weight, yet more preferably 0.01% to 1% by weight, further preferably 0.01% to 0.5% by weight, most preferably 0.01% to 0.3% by weight, based on the overall composition of the cement prior to hardening.

6. The water-hardening dental cement as claimed in claim 1, in that it comprises, based on the overall composition of the cement prior to hardening, one or more of the constituents mentioned hereinbelow in the quantitative proportions mentioned hereinbelow:
20% to 90% by weight, preferably 40% to 85% by weight of acid-reactive powder,
4.9% to 40% by weight, preferably 7.4% to 25% by weight of polyprotic acid, and/or
4.9% to 40% by weight, preferably 7.4% to 25% by weight of water.

7. The water-hardening dental cement as claimed in claim 1, characterized in that the acid-reactive powder is selected from metal oxides, metal hydroxides, mineral trioxide aggregate, hydroxyapatite, bioactive glasses, especially acid-reactive glasses and mixtures thereof.

8. The water-hardening dental cement as claimed in claim 1, characterized in that the acid-reactive powder is selected from a first quantity of glass particles having a mean particle size of from 5 to 20 μm, more preferably 5 to 15 μm, and a second quantity of glass particles having a mean particle size of from 1 to 5 μm, more preferably 2 to 3 μm.

9. The water-hardening dental cement as claimed in claim 1, characterized in that the polyprotic acid is selected from polyacids and phosphoric acid.

10. The water-hardening dental cement as claimed in claim 1, characterized in that the water-hardening dental cement includes further additional constituents selected from the group consisting of complexing agents, inorganic and organic fillers, inorganic and organic colorants and mixtures of these.

11. A method for producing a water-hardening dental cement, characterized in that at least an acid-reactive powder, a polyprotic acid, water and dispersed polymer particles are mixed.

12. A kit for producing a water-hardening dental cement comprising the constituents:
a) dispersed polymer particles,
b) an acid-reactive powder,
c) a polyprotic acid, and
d) water.

13. The kit as claimed in claim 12, characterized in that it comprises, based on the overall composition of the kit prior to hardening of the dental cement, one or more of the constituents mentioned hereinbelow in the quantitative proportions mentioned hereinbelow:
0.005% to 5% by weight, preferably 0.005% to 3% by weight, more preferably 0.01% to 1% by weight, yet more preferably 0.01% to 0.5% by weight, most preferably 0.01% to 0.3% by weight of dispersed polymer particles,
20% to 90% by weight, preferably 40% to 85% by weight of acid-reactive powder,
4.9% to 40% by weight, preferably 7.4% to 25% by weight of polyprotic acid, and/or
4.9% to 40% by weight, preferably 7.4% to 25% by weight of water.

14. The kit as claimed in either of claim 11, characterized in that the kit is composed of at least two components and the constituents of the kit are divided between these components.

15. The use of a water-hardening dental cement as claimed in claim 1 as a filling or as a luting cement.
